(19) United States
(12) Patent Application Publication
(10) Pub. No.: US 2007/0020636 A1
(43) Pub. Date: Jan. 25, 2007

(54) Method for early detection and monitoring of diseases by analysis of cell-surface-bound nucleic acids

(75) Inventors: Georg Sczakiel, Gross-Gronau (DE); Valentin Vlassov, Novosibirsk (RU); Pavel Laktionov, Novosibirsk (RU); Elena Rykova, Novosibirsk (RU); Svetlana Tamkovic, Novosibirsk (RU); Tat'yana Skvortsova, Novosibirsk (RU)

Correspondence Address: LARSON AND LARSON, 11199 69TH STREET NORTH, LARGO, FL, 33773, US

(73) Assignee: UNIVERSITATSKLINIKUM SCHLESWIG-HOLSTEIN

(21) Appl. No.: 10/576,005

(22) Filed: Mar. 28, 2006

Related U.S. Application Data
(63) Continuation-in-part of application No. PCT/EP04/09218, filed on Aug. 17, 2004.

(30) Foreign Application Priority Data
Aug. 18, 2003 (RU) 2003125486

(51) Int. Cl. C12Q 1/68; C12P 19/34

(52) U.S. Cl. 435/6; 435/91.2

(57) ABSTRACT

This invention relates to noninvasive methods of early detection of different sicknesses, like precancerous state or early stages of cancer development, pathologies of pregnancy, and monitoring of efficacy of anticancer therapy. The method is based on cell-surface extra-cellular nucleic acids from human blood which is divided into plasma and cellular fractions, and further divided into Leukocytes and erythrocytes. Cell-Surface-bound extra-cellular nucleic acids are eluted form cell surface with PBS-EDTA treatment or treatment of cells with trypsin solution. Eluted nucleic acids are isolated with convenient method and analyzed for presence of at least two specific sequences of nucleic acids such as PCR analysis, multiplex PCR, hybridization assay or other methods, thereby increasing the reliability of early detection of the diseases with abnormal functioning of genetic apparatus of cells due to increase of sensitivity of detection of specific DNA and RNA sequences.

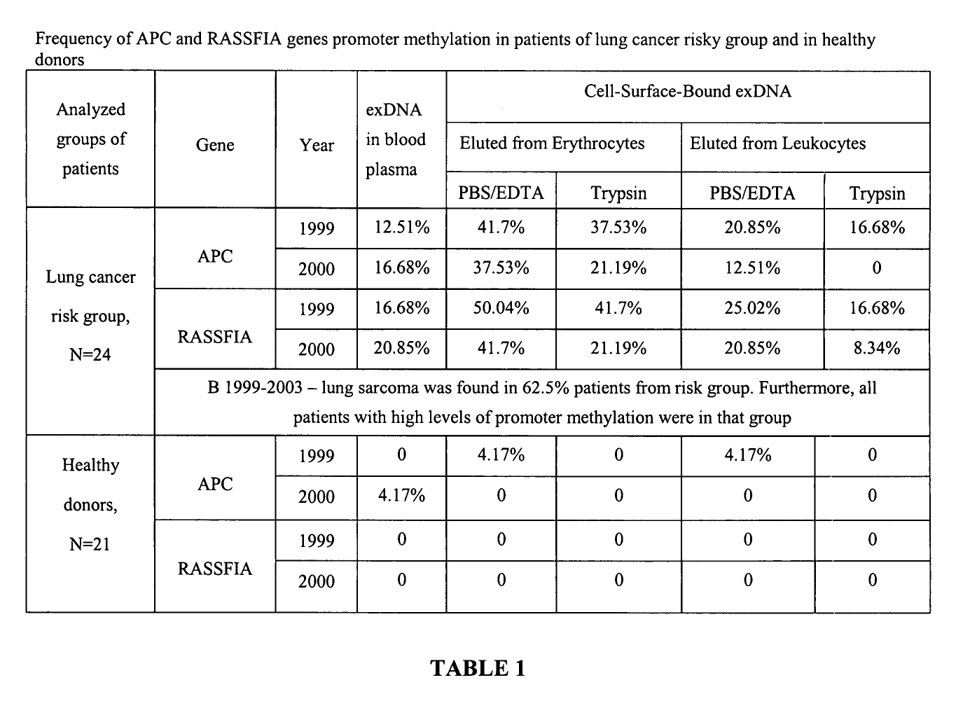

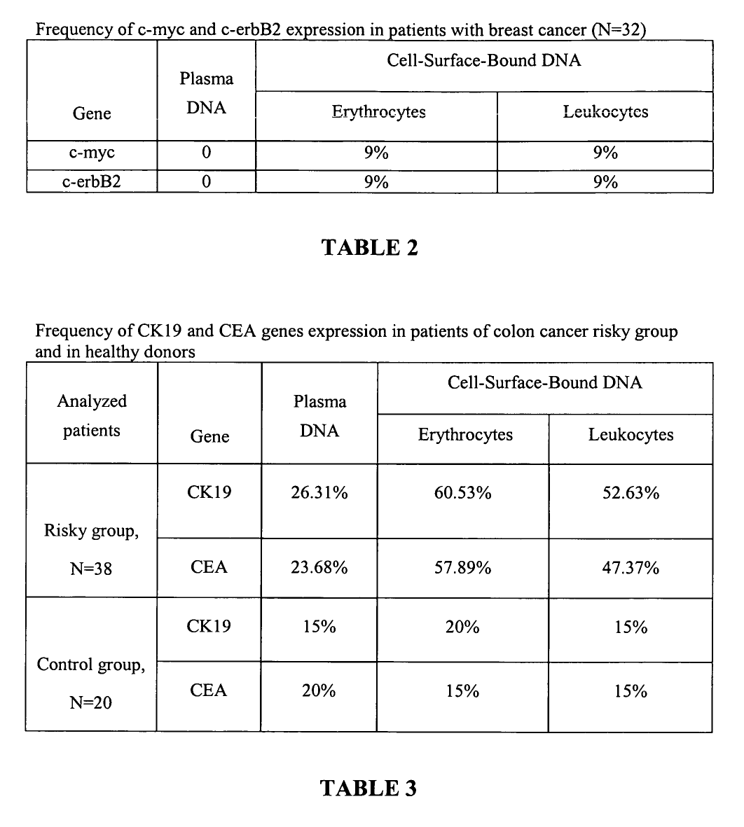

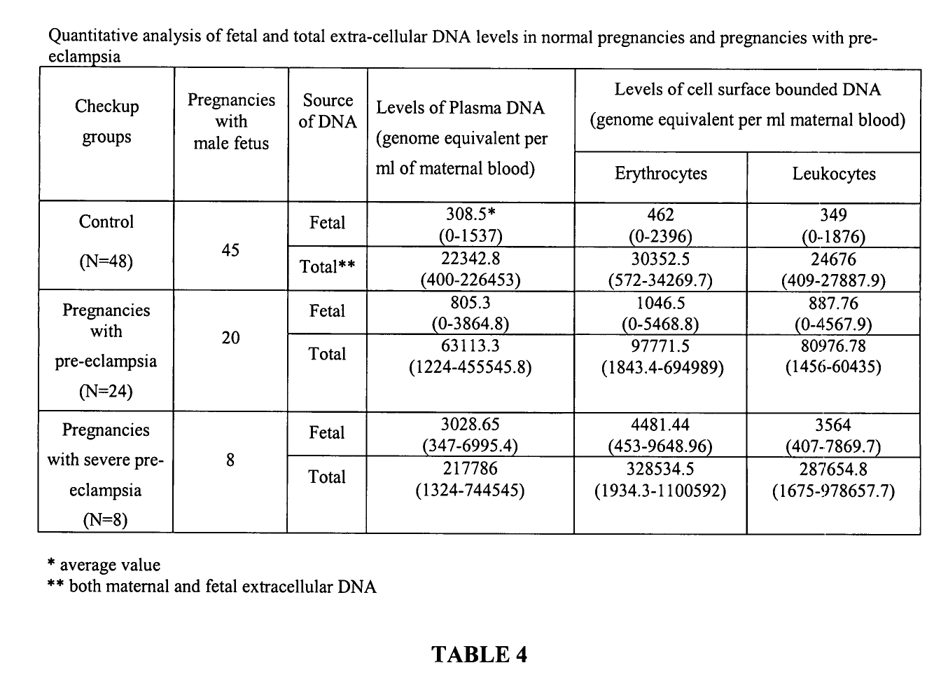

### PRIOR APPLICATIONS

This is a U.S. continuation-in-part utility patent application which bases priority on International application PCT/EP2004/009218, filed on Aug. 17, 2004, which in turn bases priority on Russian application 2003125486, filed on Aug. 18, 2003.

### BACKGROUND OF THE INVENTION

1. Field of Invention

The invention belongs to the filed of diagnostic medicine and therapy monitoring. It is based on the development of non-invasive methods for early detection of human diseases including, but not limited to, pre-cancerous states, early states of cancer development, pathologies of pregnancy and monitoring of efficacy of anticancer therapy.

2. Description of the Prior Art

There is a need for the development of non-invasive methods for early detection of human diseases, especially pre-cancerous states, early states of cancer development, pathologies of pregnancies and the monitoring of the effectiveness of anticancer therapy.

Many invasive procedures exist in the prior art, but these are no considered ideal for many patients. Although invasive procedures can more often than not provide an exact determination of an underlying human disease to an attending physician, it can be harmful to that sick person. It is desirous to minimize invasive procedures as much as possible. This is especially true in the early detection of human diseases, such as precancerous states, early stages of cancer development, pathologies of pregnancies and effectiveness monitoring of anticancer therapy. It is greatly needed to eliminate these invasive procedures all together, if possible.

### SUMMARY OF THE INVENTION

It is an object of the invention to provide a method of early detection and monitoring of diseases. It is also an object of the invention to provide a method for the purpose of early detection and monitoring of diseases that is non-invasive. It is another object of the invention to provide a method that allows for the early detection of cancer of different genesis. It is furthermore an object of the invention to provide a method for the early detection in the pathology or pregnancy and it is an object of the invention to provide a method for the monitoring of the efficacy of the anticancer therapy.

### BRIEF DESCRIPTION OF THE DRAWINGS

The detailed description of the invention, contained herein below, may be better understood when accompanied by a brief description of the drawings, wherein:

TABLE 1 shows the correlation between the symptom lung cancer and increased amounts of extra-cellular and cell-surface-bound nucleic acids (from leukocytes and erythrocytes). Both groups were sampled from a lung cancer risk group and healthy donors. The number in percentages show to which extent the samples were marked positive for the gene-markers “APC” and “RASSFlA”. The cell-surface-bound nucleic acids are divided into sub-cellular fractions for erythrocytes and leukocytes and further distinguished by their method of elution (PBS-EDTA or trypsin treatment);

TABLE 2 shows that the nucleotide sequences “c-myc” and “c-erbB2” are detachable in preparation of cell-surface-bound nucleic acids of leukocytes and erythrocytes in 9% of patients with breast cancer;

TABLE 3 shows that the nucleotide sequences CK19“and CEA” are detachable in preparations of cell-surface-bound nucleic acids of leukocytes and erythrocytes as well as in cell-free plasma-DNA in a colon cancer risk group; and

TABLE 4 shows an increase of DNA-concentration in plasma and of cell-surface-bound DNA of leukocytes and erythrocytes in samples of pregnant women with differing degree of pre-eclampsia.

### DETAILED DESCRIPTION OF THE PREFERED EMBODIMENT

The method according to the invention is based on the investigation of cell-surface-bound extra-cellular nucleic acids from human blood. Blood samples are divided into plasma and cellular fractions. The cellular function is further sub-divided into leukocytes and erythrocytes. Cell-surface-bound extra-cellular nucleic acids are eluted from cell surface with PBS-EDTA or by treatment of cells with trypsin solution. Eluted nucleic acids are isolated with and analyzed for the presence of at least two specific markers (nucleotide sequences) associated with disease or parameter of interest by using analytical methods such as PCR multiplex PCR, hybridization assay or other methods of investigation of specific sequences of nucleic acids.

The method enables one to increase the reliability and sensitivity of early detection of diseases or therapeutic schemes. This strategy shows improved sensitivity of the detection of specific DNA and RNA sequences in the fraction of nucleic acids associated with cell-surface of blood cells when compared with nucleic acids isolated from the plasma fraction. This is especially important for the reliable detection of early stages of pathological processes at which the most part of nucleic acids circulating in the blood are associated with cell-surface of blood cells. It is important to note that this methodology in non-invasive and thus, the potential risk by the diagnosis itself is substantially minimized when compared to invasive methods.

The method according to the invention allows the isolation of nucleic acids, obtained from the cell-surface of blood-circulating cells, as diagnostic markers:
1. A patient's blood sample is being separated into plasma and cellular fraction.
2. The cellular fraction is further divided into erythrocytes and leukocytes.
3. Nucleic acids associated with the cell surface of leukocytes are eluted by treatment with PBS-EDTA.
4. The eluted nucleic acids are isolated by methods known in the state of the art (e.g., with a kit from Qiagen or any other suitable laboratory protocol).
5. The composition of these nucleic acid preparations and the absolute and relative amounts can be analyzed with any suitable method known in the state of the art, e.g., PCR, Multiplex-PCR, hybridization and sequencing methods.

In more detail, the method of early diagnosis of diseases induced by abnormal functioning of cellular genomes comprises sampling of blood, dividing the blood into plasma and cellular fractions, isolating extra-cellular nucleic acids (exNA), reveling specific sequences of nucleic acids by means of polymerase chain reaction with subsequent analysis of the presence or absence of specific sequences in total PCR products, which differ from existing methods by the fact that cell-surface-bound extra-cellular nucleic acids are used as a source of extra-cellular nucleic acids instead of exNA isolated from plasma fraction, whereby the cellular fraction is divided into leukocytes and erythrocytes, cell-surface-bound extra-cellular nucleic acids are subsequently eluted from cell surface, exNA are isolated from elutes and these exNA are used for analysis of at least two specific sequences of exNA distinctive for the diseases.

In a preferred embodiment of the invention, the cell-surface-bound nucleic acids are eluted by treating the cells with 10 volumes of PBS with 5 mM EDTA at 4° C. with subsequent pelleting of the cells by centrifugation and collection of the supernatant, followed by the elution with 0.25% trypsin solution, subsequent inactivation of the enzyme with trypsin inhibitor, centrifugation and collection of the supernatant.

Having thus described the present invention in the detailed description of the preferred embodiment, what is desired to be obtained in Letters Patent is:

1. A method for the early diagnosis of diseases in a human induced by abnormal functioning of cellular genomes, the steps of the method comprising:
a) sampling blood of the human;
b) dividing the blood into plasma and cellular fractions;
c) isolating extra-cellular nucleic acids (exNA); and
d) revealing specific sequences of nucleic acids by means of polymerase chain reaction with subsequent analysis of the presence or absence of specific sequences in total PCR products, whereby cell-surface-bound extra-cellular nucleic acids are used as a source of extra-cellular nucleic acids instead of exNA isolated from plasma fraction, and whereby the cellular fraction is divided into leukocytes and erythrocytes, cell-bound-surface extra-cellular nucleic acids are subsequently eluted from cell surface, exNA are isolated from elutes and these exNA are use for analysis of at least two specific sequences of exNA distinctive for the disease.

2. The method according to claim 1, the steps of the method further comprising:
a) providing a two-stage elution of exNA from the surface of leukocytes;
b) eluting exNA by treatment of cells with 10 volumes of PBS supplied with 5 mM EDTA at 4° C.;
c) pelleting of cells by centrifugation and collection of supernatant;
d) eluting of exNA with 0.25% trypsin solution;
e) inactivating of an enzyme with trypsin inhibitor; and
f) centrifugation collection of supernatant.

3. The method according to claim 1, wherein exNA is isolated by means of increased glass-milk protocol.

4. The method according to claim 1, wherein early detection of cancer or pathologies of pregnancy is indicated.
